(19) United States
(12) Patent Application Publication
(10) Pub. No.: US 2004/0063150 A1
(43) Pub. Date: Apr. 1, 2004

(54) Arrangement for the production testing and archiving of chemical or biological arrays bonded to a solid phase

(76) Inventors: Gunter Mayer, Jena (DE); AndreasArne Schober, Schwangau (DE); Arne Brecht, Ilmenau (DE); Thomas Henkel, Jena (DE); Matthias Kallenbach, Stutzerbach (DE)

Correspondence Address: Jordan and Hamburg, 122 East 42nd Street, New York, NY, 10168, US

(21) Appl. No.: 10/416,944

(22) PCT Filed: Nov. 30, 2001
(86) PCT No.: PCT/EP01/13999

(30) Foreign Application Priority Data
Nov. 30, 2000 (DE) 100 60 502.8

(51) Int. Cl. G01N 33/53; C12M 1/34; G01N 33/543

(52) U.S. Cl. 435/7.1; 436/518; 435/287.2

(57) ABSTRACT

The present invention relates to an arrangement for producing, testing and archiving chemical or biological arrays bonded to a solid phase.

The aim of the invention is to define such an arrangement which guarantees a locally-defined, temporary binding of individual solid phase supports to a common base support, and simultaneously permits a transformation of the individual solid phase supports, according to a protocol, whereby any complete combined array or a subset thereof can be generated. Said aim is achieved, when the support is formed from a large-surface support plate with a planar or arched surface form. Said base support (2) is provided with a number of discrete regions of elevated magnetic field strength, each of which permits a removable attachment and retention of a solid phase support (1), each comprising a corresponding magnetic or magnetizable element (11), associated with a region of elevated magnetic field strength, such as a magnetic element (21) on the base support (2). The curvature of the surface of the solid phase support (1), facing the base support surfaces, matches the curvature of the base support surface to give a flush fit and the solid phase support (1) facing away from the base support (2) surface is provided with a functional layer (12) for the permanent bonding of chemical or biological substances.

### BACKGROUND OF THE INVENTION

The present invention relates to an arrangement for producing, testing and archiving chemical or biological arrays bonded to a solid phase, whereby the solid phase supports are provided with at least one defined compound. The arrangement recommended makes it possible that the individual solid phase supports assigned to a base support can be transported, archived and analyzed with the base support and can be caused to react with dissolved gaseous chemical reagents.

The arrangement is preferably used for the production, administration and manipulation of substance arrays bonded to solid phases in order to characterize the individual compounds (atomic elements of the array) fixed to solid phase supports with respect to their molecular interaction with complex biological, biochemical, physiological and other systems and to select and identify compounds exhibiting specific features. In addition to this, this arrangement can be used for all processes which require the defined distribution of molecular or biological or biochemical properties to multiple elements, whereby the manipulation of individual elements or element arrays is possible.

According to the prior state of technology, substance arrays are produced

1) either in the conventional split-and-mix procedure. On the one hand, this technique ensures that each function support carries a defined, uniform compound and the number and structure of all compounds being in the array are known. On the other hand, the chemical information cannot be assigned to one specific particle. This assignment is only possible by performing a subsequent analysis. The use of coded function supports combined with a sample tracking system allows to identify synthesis supports and to record the operations and manipulations performed with it. In this way it is possible to assign chemical information to the synthesis support. Due to the combination of a synthesis support with a code system, such arrangements cannot be used for miniaturization, that is for single synthesis resin particles which have a diameter of between 20 and 200 μm in general and of between-10 and 2000 μm in special kinds of applications.

2) or by means of the conventional synthesis of the elements of the array in defined single compartiments which are processed individually. In this way, a clear assignment of a compartiment and a chemical structure is possible. The degree of parallelization obtainable by this procedure is comparatively low. Therefore, the synthesis of a typical complete combined array including ca. 10⁶ to 10⁹ elements cannot be performed according to the prior state of technology.

The different types of titer plates known according to the state of technology do not allow a reliable fixation of the substance and/or reaction support units. Common reaction and synthesis systems with a local fixation (e.g. with localized support substances) show a number of disadvantages when handling the substance and/or reaction support units.

The aim of the present invention is to define an arrangement for producing, testing and archiving chemical or biological arrays bonded to a solid phase which ensures a locally defined, temporary binding of individual solid phase supports to a common base support and simultaneously allows a transformation of the individual solid phase supports according to the protocol, whereby any complete combined array or a subset thereof can be generated.

For the present invention the base support is formed from a substrate provided with a surface geometry and structure adjusted to the application. Said base support is provided with a magnetic element which generates a defined structured magnetic field at least on one of the support surfaces. Elements the geometry of which is adjusted to the appropriate application are used as solid phase supports which can be processed. These elements are provided with inducable or permanent magnetic properties showing a typical maximum dimension (length, width) of between 50 μm-5000 μm, in particular cases between 20 μm-10000 μm, whereby the solid phase supports are additionally provided with at least one material for the chemical immobilization of chemical compounds or with suitable compartiments for the physical immobilization of substances. Manipulators according to the present invention are equipment units and devices which can be used for loading the base support with the individual solid phase supports and to place and pick up an individual or several solid phase supports. The specific design of these manipulators can vary and is not part of the present invention.

The base support taking up the solid phase supports preferably includes permanent magnets which are arranged and/or structured in such a way that the overlaying of all the magnetic partial fields leads to a magnetic field with a large number of point-like and linear regions of highest field strength which are distributed over the total area of the matrix of the solid phase supports to be fixed.

### DETAILED DESCRIPTION OF THE INVENTION

The present invention will now be described in more detail by way of the following schematic example. The figures show:

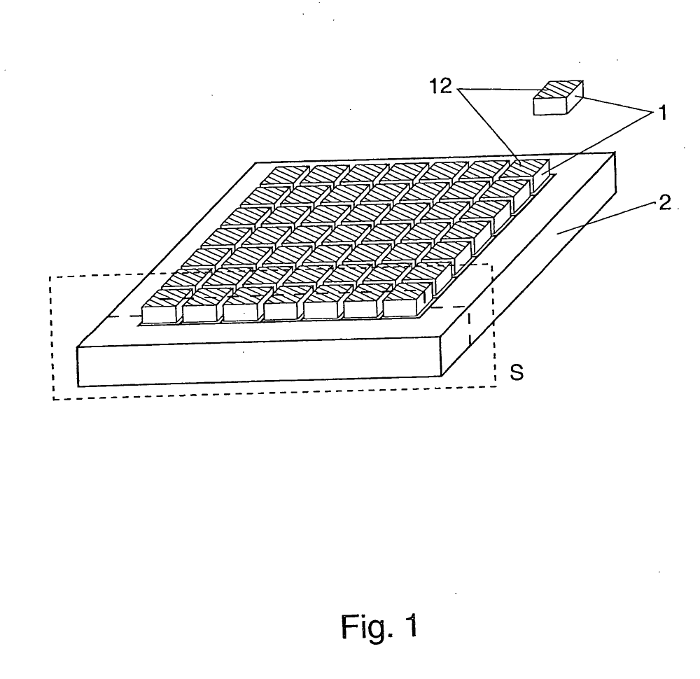

FIG. 1 an example of a base support being loaded with several individual solid phase supports in perspective view, and

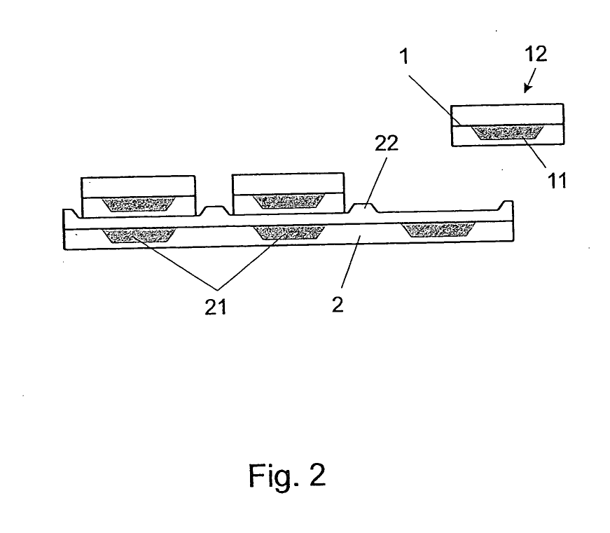

FIG. 2 a section through a possible formation of the base support and of the solid phase supports fixed on it.

Two kinds of basic functional elements are required for the arrangement recommended. The first ones are solid phase supports 1 (substance supports), in the given example being formed by little wafers with a dimension ranging from some micrometers to several millimeters. At least on one part of their surface, these wafers are modified or coated in such a way that chemical or biological substances can be bonded permanently or temporarily. The second element is a base support 2 formed from a large-surface support plate with a preferably planar surface on which the solid phase supports 1 can be arranged in a defined geometry and can be reversibly fixed, as shown in FIG. 1.

This fixation is due to magnetic forces. Comparing the fixation by mechanical friction or form fitting with the magnetically caused fixation the latter one offers the advantage that functionally required cavities or undercuts possibly leading to substance entrainment in chemical processes in solutions do not exist.

FIG. 2 is a section along a plane S according to FIG. 1, whereby a solid phase support 1 is shown in raised position. The solid phase supports 1 and the base support 2 include magnetic elements the interaction of which causes the fixating force. At least the magnetic elements 21 of the base support 2 are permanently magnetized. The magnetic elements of the solid phase support 1 are either also permanently magnetized or they are ferromagnetic and will be magnetized temporarily in the field of the base support 2. If the solid phase supports 1 contains ferromagnetic elements 11, they are designed in such a way that the center of mass of the ferromagnetic elements is located close to the contact surface to the base support to develop a preference orientation on the base support plate. The effect made use of is the attracting magnetic force between bodies having the same magnetizing orientation. The magnetic elements in the base support 2 are magnetized in a unipolar manner perpendicular to the fixation plane. The magnetic elements 11 of the solid phase supports 1 are or will be magnetized in a perpendicular orientation to the contact surface. The magnetic elements 21 of the base support are arranged in such a way that the local maxima of the field strength and of the field strength gradient (the product of both parameters is decisive for the value of the force effect) are at the geometric locations at which the solid phase supports are to be fixated. The shape and dimension of the magnetic elements 11 in the solid phase supports 1 and in the base support 2 are selected as to ensure that the solid phase supports 1 are reliably fixed despite the interactions between adjacent elements. Preferably, the magnetic elements are formed by single magnetic bodies being inserted into recesses of the base support plate. The magnetic elements can also be formed by a magnetic material layer showing a local concentration of magnetic material or by a mostly homogeneous magnetic material layer locally showing an increased or alternating magnetization. Other kinds of forming the magnetic elements in the support can also belong to the present invention. Generally, it is important that the base support contains multiple regions of an elevated magnetic field strength which allows the fixation of the solid phase supports 1. Depending on the kind of application of the arrangement recommended it is also possible that magnetic elements 21 being adjacent on the lines of the base support 2 are combined to one line. To protect the magnetic elements 21 of the base support 2 against corrosion they are provided with a protective coating or they are completely surrounded or covered by the basic material of the base support 2 or by the side of the solid phase supports 1 facing the base support. Mechanical auxiliary units 22 can be added to support the manipulation. Preferably, these auxiliary units are flat elements in a conic or pyramid-like shape located on the contact surface of the base support 2. They cause a rough orientation of the solid phase supports 1.

Silicon, glass or a chemically stable plastic material is to be used preferably for the base support 2 and for the solid phase supports 1.

FIG. 2 illustrates a preferred version using silicon as material. In the example, both the solid phase supports 1 and the base support 2 consist each of two silicon wafers. One of the two wafers of each support is provided with recesses by wet-chemical etching. These recesses are filled with magnetic or magnetizable material, preferably in a screen printing process The upper silicon wafers always cover the lower ones. The upper plane of the solid phase supports 1 is provided with a functional layer 12 for the permanent bonding of chemical or biological substances. In the example, the base support 2 is provided with additional structures 21 for supporting the manipulation. These structures can also be produced by wet-chemical etching in the upper plane of the silicon wafer covering the base support 2.

The elements 1, 2 mentioned can also be built up from one substrate plane covered by a protective layer. Plastic materials being resistant to chemicals, such as fluoroplastics or polyimides or organic-inorganic composite materials, are preferably used for the protective layer.

It is also possible to use glasses/ceramics as a basic material. They are structured by casting or embossing techniques or by removing processes such as etching, laser cutting or sand blasting. An extremely advantageous version will be obtained, if plastic materials resistant to chemicals are used as the basic material and the magnetic elements are embedded into the base support by injection molding.

The use of the described arrangement for the production of a combined array will now be explained in more detail, by way of example.

This example relates to the synthesis of a complete combined peptide array, whereby the substances alanine, asparagine and glycine are used, whereby Fmoc protected pentafluorophenylesters of the amino acids are used.

First, three base supports 2 according to FIG. 1 are used. Nine solid phase supports 1 are fixed on each of them. Moreover, three reagent-specific immersion baths are required for the synthesis operations. All base supports 2 use the immersion baths for protection-removing and washing steps. After every synthesis step the solid phase supports 1 are re-organized on the three supports according to the protocol.

In a first reaction vessel all solid phase supports 1 fixed to the first base support 2 react with Fmoc-Ala-OPfp; in a second reaction vessel b all solid phase supports 1 fixed to the second base support 2 react with Fmoc-Asn-OPfp and in the third reaction vessel c all the solid phase supports 1 fixed to the third base support 2 react with Fmoc-Gly-OPfp.

| | first base | second base | third base |
|---|---|---|---|
| | support | support | support |
| 1^{st} synthesis | in reaction | in reaction | in reaction |
| step | vesseL a: | vessel b: | vessel c: |
| | Ala | Asn | Gly |
| | Ala | Asn | Gly |
| | Ala | Asn | Gly |
| | Ala | Asn | Gly |
| | Ala | Asn | Gly |
| | Ala | Asn | Gly |
| | Ala | Asn | Gly |
| | Ala | Asn | Gly |
| | Ala | Asn | Gly |

Then, three of the solid phase supports 1 are taken from each of the corresponding three base supports 2 and sorted anew on the three base supports in such a way that three solid phase supports 1 coming from another reaction vessel are fixed to each base support 2. Each of the base supports 2 is loaded with nine solid phase supports 1 anew and will be brought into one of the reaction baths a, b, c again, as you can see in the following table.

| | first base | second base | third base |
|---|---|---|---|
| Sort | support | support | support |
| 2^{nd} synthesis | in reaction | in reaction | in reaction |
| step | vesseL a: | vessel b: | vessel c: |
| | Ala-Ala | Ala-Asn | Ala-Asn |
| | Ala-Ala | Ala-Asn | Ala-Asn |
| | Ala-Ala | Ala-Asn | Ala-Asn |
| | Asn-Ala | Asn-Asn | Asn-Asn |
| | Asn-Ala | Asn-Asn | Asn-Asn |
| | Asn-Ala | Asn-Asn | Asn-Asn |
| | Gly-Ala | Gly-Asn | Gly-Asn |
| | Gly-Ala | Gly-Asn | Gly-Asn |
| | Gly-Ala | Gly-Asn | Gly-Asn |

Analog to the second synthesis step, the individual solid phase supports 1 are again removed from the corresponding base supports and newly sorted in a defined manner onto the base supports 2. See table below for the third synthesis step following:

| | first base | second base | third base |
|---|---|---|---|
| Sort | support | support | support |
| 1^{st} synthesis | in reaction | in reaction | in reaction |
| step | vesseL a: | vessel b: | vessel c: |
| | Ala-Ala-Ala | Ala-Ala-Gln | Ala-Ala-Gly |
| | Asn-Ala-Ala | Asn-Ala-Gln | Asn-Ala-Gly |
| | Gly-Ala-Ala | Gly-Ala-Gln | Gly-Ala-Gly |
| | Ala-Asn-Ala | Ala-Asn-Gln | Ala-Asn-Gly |
| | Asn-Asn-Ala | Asn-Asn-Gln | Asn-Asn-Gly |
| | Gly-Asn-Ala | Gly-Asn-Gln | Gly-Asn-Gly |
| | Ala-Asn-Ala | Ala-Asn-Gln | Ala-Asn-Gly |
| | Asn-Asn-Ala | Asn-Asn-Gln | Asn-Asn-Gly |
| | Gly-Asn-Ala | Gly-Asn-Gln | Gly-Asn-Gly |

As demonstrated in the table above, all possible synthesis results of the simple example given are obtained. The special advantage of the described arrangement will be achieved, if multiple solid phase supports 1 are fixed on appropriately larger base supports 2.

This invention offers considerable advantages compared to the synthesis of chemical or biological arrays which are performed according to the currently common array-based procedures which preferably use micro-titer plates for the synthesis of arrays, whereby the chemical functional supports (beads) are added in cavities of titer plates, whereby the reagents are to be dispensed to the single cavities during the synthesis operations, whereby problems arise, in particular reagent-dependent differences in reaction times, incompatibilities of different reagents with one another and the performance of specific adding modes.

The application of arrangements following this invention, in which the supports described together with solid phase supports temporarily immobilizable on them are used for the synthesis of substance arrays, allows a drastic reduction in all liquid-handling operations and an increased process reliability of chemical syntheses. The solid phase supports are combined in an optimum way on a base support for each step of the synthesis. Together with the solid phase supports these base supports are put into a reaction vessel best suited for the corresponding synthesis operation. Thus, pipette operations which require much effort, particularly when using humidity- or air-sensitive reagents, are not necessary. Consequently, this inventive solution offers a considerable potential for saving time and money. In addition to this, the arrangement allows to perform synthesis steps in especially adjusted reaction vessels. Thus, the range of chemical reactions available for the synthesis of substances becomes wider, and therefore the diversity and quality of synthesized substance arrays increase. Another important advantage of the arrangement presented is given in the fact that the base supports loaded with solid phase supports can be transported, archived and analyzed without any problem and can be caused to react with dissolved gaseous chemical reagents with the option to set the temperatures and pressures in the reaction room according to the individual demands.

### List of Reference Numerals

1—solid phase support

11—magnetic or magnetizable element

12—functional layer

2—base support

21—magnetic element of the base support 2

22—mechanical auxiliary units

S—cutting plane

1. Arrangement for production, testing and archiving chemical or biological arrays bonded to a solid phase, consists of a base support (2) formed by a large-surface support plate with a planar or curved surface and said base support (2) is provided with a number of discrete regions with an elevated magnetic field strength, each of which allows a removable attachment and retention of a solid phase support (1), each comprising a magnetic or magnetizable element (11) associated with a region of elevated magnetic field strength such as a magnetic element (21) on the base support (2) whereby the curvature of the surface of the solid phase support (1) facing the base support surface, matches the curvature of the base support surface to give a flush fit and the solid phase supports (1) facing away from the base support (2) surface are provided with a functional layer (12) for the permanent bonding of chemical or biological substances.

2. Arrangement according to claim 1, wherein the solid phase supports (1) provided with ferromagnetic elements (11) are formed in such a way that the mass center of the ferromagnetic elements (11) is located near the contact surface of elevated magnetic field strength of the base support (2).

3. Arrangement according to claim 1, wherein the regions of elevated magnetic field strength of the base support (2) are completely provided with a corrosion-resistant protective layer or with an extra cover.

4. Arrangement according to claim 1, wherein the base support (2) is provided with mechanical auxiliary units (22) realized as flat conic or pyramid-like elevated elements between the regions of increased magnetic field strength for easing the adjustment of the solid phase supports (1).

5. Arrangement according to claim 1 or 2, wherein the solid phase supports (1) are formed from at least one half-shell for taking up a magnetic or magnetizable element (11) and from a cover which covers the half-shell completely and tightly and which is provided with a functional layer (12) on the surface facing away from the element (11).

6. Arrangement according to anyone of the preceding claims, wherein the base support (2) and the solid phase support (1) are mainly made of silicon, glass, ceramics or a chemically stable plastic material.

7. Use of an arrangement according to the preceding claims, wherein for the production of completely combined substance arrays portions of the solid phase supports (1) are removed from first base supports (2) according to a predefined protocol after a completed synthesis step and which, in a further synthesis step, are then partly resorted on other base supports containing solid phase supports (1) from another preceding synthesis step, whereby this procedure as well as the normal intermediate washing steps are repeated as often as the desired substance arrays are obtained.
